(19) United States
(12) Patent Application Publication
(10) Pub. No.: US 2023/0173163 A1
(43) Pub. Date: Jun. 8, 2023

(54) FEMININE CARE DEVICE

(71) Applicant: Anita Nischal, Old Bridge, NJ (US)

(72) Inventor: Anita Nischal, Old Bridge, NJ (US)

(21) Appl. No.: 17/977,553

(22) Filed: Oct. 31, 2022

Related U.S. Application Data
(60) Provisional application No. 63/274,243, filed on Nov. 1, 2021.

(51) Int. Cl. A61M 3/02; A61M 31/00; A46B 11/00; A46B 9/02

(52) U.S. Cl. CPC A61M 3/0279; A61M 3/0262; A61M 31/00; A46B 11/0055; A46B 9/026; A61M 2210/1475; A46B 2200/1006

(57) ABSTRACT

A feminine care device comprising an elongated cylinder having a tapered tip, a base portion, and a hollowed interior where the cylinder forms a shape of a bullet. A plurality of bristles is coupled to and covers a substantial portion of the cylinder. A cylindrical shaped plunger has a hollowed interior for storing liquid, where the plunger is configured to be assembled within and disassembled from the hollowed interior of the cylinder. A plurality of slits is formed in the cylinder for passing liquid stored in the plunger.

### CROSS-REFERENCE TO RELATED APPLICATIONS

This application claims priority to U.S. Provisional Application No. 63/274,243, filed Nov. 1, 2021, which application is hereby incorporated in its entirety by reference.

### TECHNICAL FIELD

The present disclosure generally relates to a feminine care product, and more specifically, to a brush that removes excessive vaginal secretions.

### SUMMARY

The present disclosure is directed to a feminine care device. In particular, this device is a brush that removes excessive vaginal secretions. It is directed to women of all ages, specifically targeting the onset of menarche and throughout their lives. The objectives of this product are the following:
1. Removing excess vaginal discharge;
2. Use at the end of the menstrual cycle to remove the build-up blood which causes vaginal odor;
3. Use to remove the build-up of vaginal medications such as suppository and creams;
4. Use on the onset of vaginal discomfort from vaginal yeast and vaginal bacteria to prevent an infection;
5. Will serve to ease vaginal discomfort during the onset of symptoms;
6. Use as a devise to aid in biofilm disruptors, such as oil application;
7. Use in vaginal dryness to apply natural lubricants such as coconut oils for post menopausal women;
8. Maintains vaginal PH by removing excess discharge;
9. Prevents chronic vaginal wetness and discomfort;
10. Aids in the prevention of vaginal odor;
11. This vaginal brush will serve to replace the need for a woman to use any form of a liquid douche to clean the internal vaginal cavity since this brush will be so easy to use daily. The brush needs to be wet with water for easy insertion and rotated 1-2 times to remove any excess discharge pooling high in the vaginal vault. There will be no need to introduce any fragrances or chemical washes contained in Douche products;
11. The brush does not disrupt the vaginal bacteria or PH since it's simply tap water which comes into contact with the vaginal cavity; and
12. The softness of the bristles will also allow for cleaning the external vaginal anatomy by getting into the crevices of the labial folds—much superior to a harsh wash cloth or the roughness on the hands. This will control odor.

This devise is the first of its kind, nothing like this currently exists on the market today.

This device will put women in control of their vaginal health, allowing them to have an intimate awareness of their vaginal discharge during different phases of their menstrual cycle and sexual health, as well as being able to have control over restoration of common disruptions in the vaginal PH which causes minor discomfort that is easily addressed with simple home remedies to restore the vaginal PH.

This device allows a woman a glimpse into the vaginal cavity much like their Gynecologist who is able to differentiate an infection from a normal vaginal discharge.

One of the most common questions asked by women is How do I know if the discharge is normal. By becoming familiar with her discharge on a daily basis, this question will no longer create insecurity for women.

This device allows for the delivery of certain oils to destroy biofilms of certain types of bacteria which becomes resistant to traditional medications. This creates chronic distress and discomfort for women today. Presently their is no available medications to break biofilms in the vagina. This device will offer that hope of eradicating vaginal biofilm and restoring vaginal PH.

The disclosure herein is a summary of the invention and not an extensive overview of all contemplated embodiments. It should be appreciated that many other features, applications, embodiments, implementations and/or variations of the disclosed technology will be apparent from the accompanying drawings and from the following detailed description. While multiple implementations are disclosed, the drawings and detailed description are to be regarded as illustrative in nature and not restrictive.

### BRIEF DESCRIPTION OF THE DRAWINGS

This disclosure may be better understood by referring to the following figures. The components in the figures are not necessarily to scale, emphasis instead being placed upon illustrating the principles of the disclosure. In the figures, the reference numerals designate corresponding parts throughout the different views.

FIG. 1 is a front perspective view illustrating one example of a feminine care device according to the teachings of the present disclosure.

### DETAILED DESCRIPTION

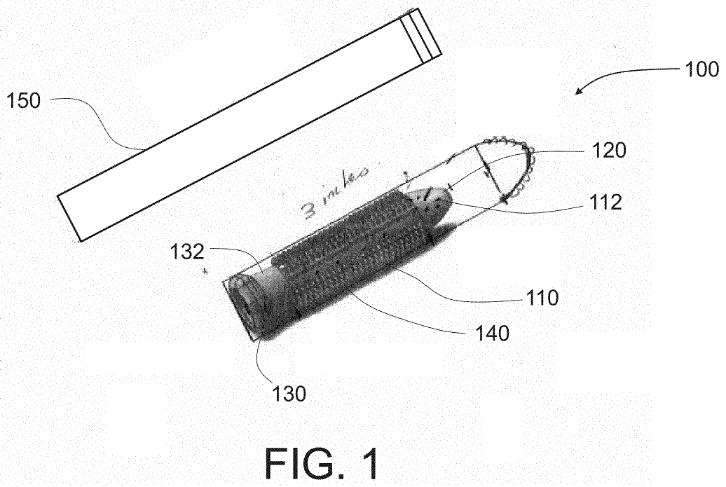

FIG. 1 illustrates one example of an implementation of a feminine care device. As described herein, the device is a cylindrical shaped hand-held unit with a comfortable grip and small tapered tip for ease of vaginal entry. Multiple small, short tapered smooth soft bristles cover a substantial portion of the cylinder. There is an ergonomic base of the cylinder for easy grip easy to rotate in the vaginal cavity.

FIG. 1 is a front perspective view of one example of a feminine care device 100 according to the teachings of the present disclosure. As shown, the device 100 includes an elongated cylindrical shaped body 110 having a tapered tip 120 formed at one end of the body 110, and a base portion 130 formed at an opposite end of the body 110. The body 110, together with the tip 120 and the base portion 130 may resemble the shape of a bullet. The various components of the device may be made of plastic, ceramic, polyvinyl chloride (PVC), medical-grade silicone for softness, or any other suitable medical-grade material.

The device 100 further includes a plurality of small short tapered smooth soft bristles 140 covering a substantial portion of the body 110. In some implementations, the bristles may cover seven centimeters of the body 110.

The ergonomical base portion 130 of the body 110 forms a grip 132 that may be engaged by a user to rotate the body 110 in a vaginal cavity.

In some implementations, the diametrical dimensions of the body 110 may be 1.3 centimeters and the length may be 9 centimeters.

A plurality of slits or holes 112 are formed in the walls of the body 110 to prevent a vacuum effect when the device is inserted into the vaginal cavity.

The interior of the body 110 may be hollowed, allowing for the use of a second piece plunger 150 to deliver natural oils and medications to the vaginal cul-da-sac and the entire vaginal canal, including the vaginal sidewalls.

The plunger 150 is a separate cylindrical shaped unit configured to fit within the hollowed interior of the body 110. The plunger 150 may be constructed to a total length of 12.7 centimeters to extend the body 110 an additional 3.7 centimeters. The plunger 150 locks in place with rotation grove and screw in place as an extension arm to lengthen the body 110, or it can smoothly be pushed straight up to fit the hollowed interior to deliver medications and oils for therapeutic use.

The medications and oils may be delivered from the hollowed interior via the slits and holes 112 and applied to vagina with the assistance of the bristles 140.

The body 110 and plunger 150 maybe be easily disassembled for purposes of cleaning the components. In addition, the body 110 and plunger 150 may be easily transported in a carrying case. The body 110 and plunger 150 may be used in tandem or alone.

It is preferred to package the device in a small case with power cord and UV light to prevent bacteria growth. Another option is to tote the device in a small cylinder case, resembling a lipstick carrying case.

The recommended use will be daily in the shower or in a private space where there is a sink to wet the device before insertion and wash the device after use.

It is recommended to replace the device monthly, or replace the base unit after a number of uses.

The device may be easy to clean with soap and water, and air dried or towel dried before placing in the UV case.

The device may remain in the shower between uses or can be portable in the same carrying case.

The device may be made in many vibrant colors.

All components of the device will be made from materials free of any chemical byproduct and safe for insertion into a body cavity.

What is claimed is:

1. A feminine care device comprising:
an elongated cylinder having a tapered tip, a base portion, and a hollowed interior, the cylinder forming a shape of a bullet;
a plurality of bristles coupled to and covering a substantial portion of the cylinder;
a cylindrical shaped plunger having a hollowed interior for storing liquid, the plunger being configured to be assembled within and disassembled from the hollowed interior of the cylinder; and
a plurality of slits formed in the cylinder for passing liquid stored in the plunger.
